(19) United States
(12) Patent Application Publication
(10) Pub. No.: US 2025/0389725 A1
(43) Pub. Date: Dec. 25, 2025

(54) METHOD FOR DETECTION OF SULFUR-CONTAINING AMINO ACID AS BIOMARKER FOR GLIOBLASTOMA USING NOVEL FLUORESCENT PROBE AND FLUORESCENCE-GUIDED SURGERY IMAGING APPLICATION THEREOF

(71) Applicant: UNIVERSITY-INDUSTRY COOPERATION GROUP OF KYUNG HEE UNIVERSITY, Yongin-si (KR)

(72) Inventors: Do-Kyoung KIM, Seoul (KR); Jae-Hoon Kim, Seoul (KR); Young-Woong Kim, Seoul (KR)

(73) Assignees: UN, (omitted); UNIVERSITY-INDUSTRY COOPERATION GROUP OF KYUNG HEE UNIVERSITY, Yongin-si (KR)

(21) Appl. No.: 19/307,220

(22) Filed: Aug. 22, 2025

Related U.S. Application Data
(63) Continuation of application No. PCT/KR2024/002403, filed on Feb. 23, 2024.

(30) Foreign Application Priority Data
Feb. 24, 2023 (KR) 10-2023-0025062
Feb. 23, 2024 (KR) 10-2024-0026529

(51) Int. Cl. G01N 33/68; C07D 413/12; C09K 11/07; G01N 21/64

(52) U.S. Cl. CPC G01N 33/6815; C07D 413/12; C09K 11/07; G01N 21/6428; C09K 2211/1033; G01N 2021/6439

(57) ABSTRACT

The present invention relates to a fluorescent probe compound for detecting cysteine and homocysteine, and a use thereof. With the ability to detect and target cysteine and homocysteine present in tumors and cancers within a short time without interference from other biomolecules, the fluorescent probe compound produced according to the present invention is highly useful in fluorescence-guided surgery and can be advantageously used in fluorescence-guided surgery for the removal of tumors and cancers including glioblastoma.

### CROSS-REFERENCE TO RELATED APPLICATION

This application is a continuation application of International Application No. PCT/KR2024/002403 filed on Feb. 23, 2024 which claims the benefit of priority from Korean Patent Application No. 10-2023-0025062 filed on Feb. 24, 2023 and Korean Patent Application No. 10-2024-0026529 filed on Feb. 23, 2024, and designating the U.S., the entire contents of which are incorporated herein by reference.

### TECHNICAL FIELD

The present disclosure relates to a compound based on 4-chloro-7-nitrobenzofurazan, a fluorescent probe including the compound, a method for selectively detecting cysteine and homocysteine using the fluorescent probe, and a use of the compound as a contrast agent for fluorescence-guided surgery of tumors and cancers including glioblastoma.

### BACKGROUND ART

Glioblastoma is the most common malignant brain tumor, and is a very fatal disease in humans by accounting for approximately 57% of gliomas and 48% of malignant central nervous system tumors. In addition, since the incidence of glioblastoma increases with age and the glioblastoma has the characteristic of occurring in the brain, accurate glioblastoma tumor removal surgery through diagnosis is required. Although there is no known exact treatment for glioblastoma, the survival rate of patients may be increased through radiotherapy, chemotherapy, targeted therapy, and surgical removal. However, despite recent advances in treatment techniques, the prognosis of glioblastoma is still unstable, and long-term survival and cure has been rarely reported.

Currently, only a clear method for treating glioblastoma is surgery to directly remove the tumors. Indocyanine green (ICG), 5-Aminolevulinic acid (5-ALA), and fluorescein are the most commonly used as contrast agents for fluorescence-guided surgery in clinical practice to remove tumors and cancers. However, since the contrast agent is administered to patients before surgery, the contrast agent may cause various side effects due to the inherent toxicity of the contrast agent and complications after surgery. In addition, in fluorescence-guided surgery, when the contrast agent does not show a clear difference between the tumor and cancer tissues and normal tissue, the tumor may not be removed cleanly, and thus there is a high risk of recurrence due to residual tumor. Therefore, the ability to target tumor sites with high sensitivity without the following side effects and complications of the contrast agent is an essential task required in fluorescence-guided surgery of tumors and cancers including glioblastoma.

Recently, in various studies, it has been reported the potential of cysteine and homocysteine as biomarkers for tumors and cancers including glioblastoma, and it has been found that cysteine and homocysteine are closely associated with central nervous system diseases, including brain tumor, and other cancers. Therefore, selective detection of cysteine and homocysteine with high sensitivity indicates targeting tumors and cancers, including glioblastoma. In addition, this selective detection indicates the potential for the development of novel contrast agents when applied to contrast agents used for fluorescence-guided surgery. Therefore, the detection of cysteine and homocysteine is becoming increasingly necessary for removing tumors and cancers, including glioblastoma.

Meanwhile, fluorescent molecular probes are organic compounds that have been utilized in various studies. The fluorescent molecular probes have been used to analyze the properties or structures of small molecular units and proteins, and have the ability to elucidate specific biological phenomena and detect disease factors. In particular, the fluorescent molecular probes have an excellent ability to detect biomolecules including biomarkers present in a living body, and may visually display fluorescence signals through detection to provide convenience. The importance of research in the field of fluorescent molecular probes is increasing significantly due to the following characteristics:

To date, many fluorescent molecular probes capable of detecting sulfur-containing amino acids, including cysteine and homocysteine, have been developed, but there are a few cases of application to the human body due to the toxicity of the fluorescent molecular probes themselves. When applied to the human body, the fluorescent molecular probes rarely have a high targeting ability for tumors or cancers. In addition, there have been rarely reported fluorescent molecular probes that have shown potential to replace contrast agents for fluorescence-guided surgery used for surgery to remove tumors and cancers based on low toxicity.

Accordingly, the present inventors developed a fluorescent molecular probe capable of selectively detecting a biomarker of glioblastoma based on a simple synthetic method, confirmed the applicability of the compound as a contrast agent for fluorescence-induced surgery for removing tumors and cancers including glioblastoma, and then completed the present disclosure.

### DISCLOSURE

### Technical Problem

An object of the present disclosure is to provide a compound for a fluorescent molecular probe capable of selectively detecting cysteine and homocysteine, which are biomarkers of tumors and cancers including glioblastoma, and a method for producing the same.

Another object of the present disclosure is to provide a fluorescent probe for detecting cysteine or homocysteine including the compound, a composition for detecting cysteine or homocysteine and a detection method using the same, and a composition for diagnosing diseases associated with cysteine or homocysteine.

Yet another object of the present disclosure is to provide an imaging composition as a contrast agent alternative for monitoring fluorescence-guided surgery that may be used in surgery to remove tumors and cancers including glioblastoma using the compound.

Yet another object of the present disclosure is to provide a fluorescence-guided surgery method for removing tumors and cancers including glioblastoma by providing a fluorescent molecular probe capable of replacing a contrast agent for fluorescence-guided surgery that is suitable for the human body without side effects based on low toxicity.

### Technical Solution

In order to achieve the object,
the present disclosure provides a compound represented by the following Chemical Formula 1:

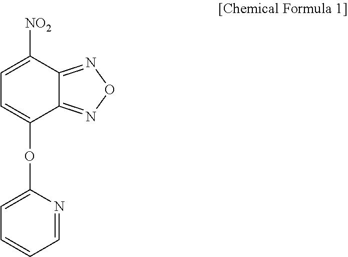 [SMILES: O=[N+]([O-])c1ccc(Oc2ccccn2)c2nonc12]

In one embodiment, the compound may react with cysteine or homocysteine to exhibit fluorescence turn-on.

In one embodiment, the compound may not emit fluorescence before contact with cysteine or homocysteine, but may emit fluorescence by contact with cysteine or homocysteine.

In one embodiment, the compound may exhibit fluorescence turn-on under conditions of pH 6.5 to 8.

In one embodiment, the compound may selectively emit fluorescence for cysteine or homocysteine, that is, selectively detect cysteine or homocysteine in a sample containing at least one selected from the group consisting of biothiols, metal ions and biomolecules.

In one embodiment, the compound may selectively emit fluorescence for cysteine or homocysteine by targeting tumors or cancers associated with cysteine or homocysteine.

In one embodiment, the tumors or cancers associated with cysteine or homocysteine may be tumors or cancers caused by the overexpression of cysteine or homocysteine, and may be, for example, at least one selected from the group consisting of brain tumor, colon cancer, stomach cancer, liver cancer, breast cancer, lung cancer, pancreatic cancer, gallbladder cancer, thyroid cancer, parathyroid cancer, kidney cancer, bladder cancer, prostate cancer, ovarian cancer, rectal cancer, central nervous system tumor, spinal cord tumor, cervical cancer, blood cancer, melanoma, colorectal cancer, bone cancer, skin cancer, head cancer, uterine cancer, vaginal cancer, vulvar carcinoma, esophageal cancer, small intestine cancer, and adrenal cancer. Preferably, the tumors or cancers may be at least one selected from the group consisting of brain tumor, liver cancer, cervical cancer, and skin cancer, more preferably brain tumor, and most preferably glioblastoma.

In addition, the present disclosure provides a fluorescent probe for detecting cysteine or homocysteine, including a compound having a leaving group in a structure represented by the following Chemical Formula 2, a hydrate thereof, a solvate thereof, or a pharmaceutically acceptable salt thereof:

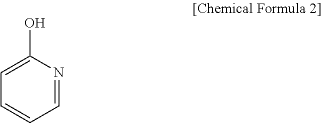

In one embodiment of the present disclosure, the compound having the leaving group in the structure represented by Chemical Formula 2 may be the compound represented by Chemical Formula 1.

In one embodiment of the present disclosure, the fluorescent probe may be able to target and image tumors or cancers associated with cysteine or homocysteine.

In one embodiment of the present disclosure, the fluorescent probe may be used in fluorescence-guided surgery for removing tumors or cancers.

Furthermore, the present disclosure provides a composition for detecting cysteine or homocysteine, including the compound represented by Chemical Formula 1, a hydrate thereof, a solvate thereof, or a pharmaceutically acceptable salt thereof.

In addition, the present disclosure provides a method for detecting cysteine or homocysteine, including treating a sample, a cell, a tissue or an organ with the compound represented by Chemical Formula 1 and irradiating an excitation light source; and measuring changes in emitted fluorescence.

In one embodiment of the present disclosure, the cell, tissue or organ may be cells, tissues or organs of tumors or cancers associated with cysteine or homocysteine.

In addition, the present disclosure provides a composition for diagnosing diseases associated with cysteine or homocysteine, including the compound represented by Chemical Formula 1, a hydrate thereof, a solvate thereof, or a pharmaceutically acceptable salt thereof.

Furthermore, the present disclosure provides an imaging composition for monitoring fluorescence-guided surgery to remove tumors or cancers associated with cysteine or homocysteine, including the compound represented by Chemical Formula 1, a hydrate thereof, a solvate thereof or a pharmaceutically acceptable salt thereof.

In addition, the present disclosure provides a fluorescence-guided surgery method for removing tumors or cancers associated with cysteine or homocysteine, including: administering to a patient the compound represented by Chemical Formula 1; illuminating a surgical site of the patient with an excitation light source to identify fluorescence emitted from the compound 1; and performing surgical resection of an area emitting the fluorescence.

Furthermore, the present disclosure provides a method for producing a compound represented by Chemical Formula 1, including reacting 4-(dimethylamino)pyridine, 2-hydroxypyridine, and 4-chloro-7-nitrobenzofurazan.

### Advantageous Effects

According to the present disclosure, a fluorescent molecular probe compound (NPO-o-Pyr) has an advantage of having a simple structure and being able to be synthesized in large quantities within a short period of time.

In addition, the fluorescent molecular probe of the present disclosure has an excellent ability to detect cysteine and homocysteine, which are known as biomarkers of cancers and tumors, and has high targeting ability, fast response speed and sensitivity for glioblastoma tumor tissues based on the characteristics of low toxicity and biocompatibility, and has high targeting ability for tumor sites when applied to a glioblastoma-xenograft mouse model as well as a glioblastoma clinical tissue sample. Therefore, the fluorescent molecular probe of the present disclosure can be used as a fluorescent molecular probe capable of replacing contrast agents for fluorescence-guided surgery used to remove tumors and cancers, including glioblastoma, by overcoming the toxicity problem of conventional contrast agents for fluorescence-guided surgery.

### DESCRIPTION OF DRAWINGS

FIG. 1 is a graph showing results of simultaneously confirming changes in absorbance (Abs) and fluorescence (Emi) when Compound 1 (NPO-o-Pyr) according to the present disclosure reacts with cysteine under an aqueous solution (phosphate-buffered saline; PBS, pH 7.4) condition.

FIG. 2 is a graph showing results of simultaneously confirming changes in absorbance (Abs) and fluorescence (Emi) when Compound 1 (NPO-o-Pyr) according to the present disclosure reacts with homocysteine under an aqueous solution (phosphate-buffered saline; PBS, pH 7.4) condition.

FIG. 3 is a graph showing results of confirming changes in fluorescence upon 430 nm excitation after reaction of Compound 1 (NPO-o-Pyr) according to the present disclosure with cysteine (Cys), homocysteine (Hcy), glutathione (GSH), and hydrogen sulfide (H₂S), respectively.

FIG. 4 is a graph showing results of confirming changes in fluorescence upon 430 nm excitation after reaction of Compound 1 (NPO-o-Pyr) according to the present disclosure with cysteine (Cys), homocysteine (Hcy), thiols, various metal ions, and biomolecules under an aqueous solution (pH 7.4) condition, respectively.

FIG. 5 is a graph showing changes in absorbance over time after reaction of Compound 1 (NPO-o-Pyr) according to the present disclosure with cysteine (Cys), homocysteine (Hcy), thiols, various metal ions, and biomolecules under an aqueous solution (pH 7.4) condition, respectively.

FIG. 6 is a graph showing fluorescence spectra over time after reaction of Compound 1 (NPO-o-Pyr) according to the present disclosure with cysteine (Cys), homocysteine (Hcy), thiols, various metal ions, and biomolecules under an aqueous solution (pH 7.4) condition, respectively.

FIG. 7 is a graph showing results of confirming the selectivity efficacy over time after reaction of Compound 1 (NPO-o-Pyr) according to the present disclosure with biothiol types such as cysteine, homocysteine, GSH, and H₂S under an aqueous solution (pH 7.4) condition, respectively.

FIG. 8 is a graph showing results of confirming the selectivity efficacy over time after reaction of Compound 1 (NPO-o-Pyr) according to the present disclosure with biothiol types such as cysteine, homocysteine, GSH, and H₂S under an aqueous solution (pH 7.4) condition, respectively.

FIG. 9 is a graph showing results of a selectivity comparison experiment between NPO-o-Pyr and NPO-m-Pyr synthesized as a control group, and a structure of NPO-m-Pyr.

FIG. 10 is a graph showing results of confirming changes in fluorescence upon 430 nm excitation after reaction of Compound 1 (NPO-o-Pyr) according to the present disclosure with cysteine and homocysteine in aqueous solutions (pH 3 to 9) having different acidity.

FIG. 11 is a graph showing results of confirming limit of detection (LOD) values when Compound 1 (NPO-o-Pyr) according to the present disclosure reacts with cysteine and homocysteine, respectively.

FIG. 12 is a graph showing results of confirming the cell viability of U87MG, Huh7, HeLa, and B16F10 by evaluating the toxicity of various leaving groups, including a leaving group (2-hydroxypyridine) constituting Compound 1 (NPO-o-Pyr) according to the present disclosure.

FIG. 13 is a graph showing results of confirming the cell viability of U87MG according to a concentration of Compound 1 (NPO-o-Pyr) according to the present disclosure.

FIG. 14 is a graph showing results of confirming the cell viability of HeLa according to a concentration of Compound 1 (NPO-o-Pyr) according to the present disclosure.

FIG. 15 is a graph showing results of confirming the cell viability of Huh7 according to a concentration of Compound 1 (NPO-o-Pyr) according to the present disclosure.

FIG. 16 is a graph showing results of confirming the cell viability of B16F10 according to a concentration of Compound 1 (NPO-o-Pyr) according to the present disclosure.

FIG. 17 is a graph showing the degree of hemolysis according to a concentration of NPO-A according to the present disclosure.

FIG. 18 is a graph showing the degree of hemolysis according to concentrations of NPO-A, a structural analogue of NPO-o-Pyr, and Compound 1 (NPO-o-Pyr).

FIG. 19 shows (a) a fluorescence tissue imaging image and (b) a fluorescence intensity graph showing results of confirming whether Compound 1 (NPO-o-Pyr) according to the present disclosure may exhibit a fluorescence turn-on characteristic through a targeting ability to a tumor site by applying Compound 1 to a glioblastoma xenograft mouse model.

FIG. 20 is a schematic diagram showing (a) an analysis process to confirm whether Compound 1 may exhibit a fluorescence turn-on characteristic through a targeting ability to a tumor site by applying Compound 1 to a glioblastoma clinical tissue in a dipping form and a spraying form.

FIG. 21 shows (b) a fluorescence tissue imaging image and (c) a fluorescence intensity graph showing results of confirming whether Compound 1 may exhibit a fluorescence turn-on characteristic through a targeting ability to a tumor site by applying Compound 1 to a glioblastoma clinical tissue in a dipping form.

FIG. 22 shows (d) a fluorescence tissue imaging image and (e) a fluorescence intensity graph showing results of confirming whether Compound 1 may exhibit a fluorescence turn-on characteristic through a targeting ability to a tumor site by applying Compound 1 to a glioblastoma clinical tissue in a spraying form.

### BEST MODE

In an aspect, the present disclosure relates to a compound represented by the following Chemical Formula 1:

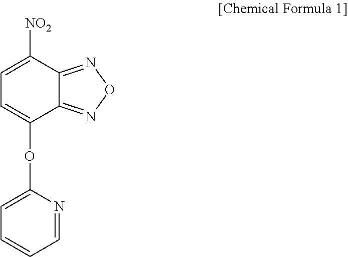 [SMILES: O=[N+]([O-])c1ccc(Oc2ccccn2)c2nonc12]

In an example of the present disclosure, the compound represented by Chemical Formula 1, 4-nitro-7-(pyridine-4-yloxy)benzo[c][1,2,5]oxadiazole, was synthesized and identified, and the compound was named ‘NPO-o-Pyr’ (see Example 1).

In one embodiment, the compound may react with cysteine or homocysteine to exhibit fluorescence turn-on.

The “fluorescence turn-on” characteristic of the present disclosure means a characteristic in which the fluorescence is not emitted in the absence (non-existence) of a target substance to be detected and is not identified with the naked eye, but the fluorescence is emitted by reacting with the target substance to be detected when the target substance to be detected is present.

In the case of a “fluorescence turn-on” reference system such as the NPO-o-Pyr compound according to an embodiment of the present disclosure, when a target substance to be detected is not present in a living tissue, a tissue isolated from a living body, or other samples, no fluorescence exists, or only weak fluorescence that is not identified with the naked eye exists, and then clear fluorescence (at least 10-fold increase in fluorescence intensity) occurs due to the presence of the target substance to be detected, so that it is possible to clearly visually determine whether the target substance to be detected is detected in a living tissue, a tissue isolated from a living body, or other samples.

The fluorescence turn-on exhibited by the compound of the present disclosure may mean that the fluorescence is not emitted before contact with cysteine and homocysteine, but the fluorescence is emitted upon contact with cysteine and/or homocysteine. Accordingly, the compound may be used as a fluorescent probe for detecting cysteine and homocysteine, may be used for targeting tumors and cancers through detection of cysteine and homocysteine, and may be used as a contrast agent for fluorescence-guided surgery.

In the present disclosure, the term ‘probe’ is also called ‘sensor’ and is defined as detecting or imaging an in vivo/ex vivo target. Generally, the probe has been used interchangeably with terms such as an imaging agent, a contrast agent, a radiopharmaceutical drug, etc.

The compound of the present disclosure may generate 2-hydroxypyridine as a leaving group after reaction with cysteine or homocysteine.

In the example of the present disclosure, since the 2-hydroxypyridine does not exhibit toxicity in various cells, it was confirmed that the 2-hydroxypyridine and the compound (NPO-o-Pyr) of the present disclosure containing the 2-hydroxypyridine as a leaving group have high biocompatibility (see Example 7). Therefore, compounds containing the 2-hydroxypyridine as the leaving group, including the compound (NPO-o-Pyr) of the present disclosure may be used as a contrast agent alternative as various fluorescent molecular probes.

In one embodiment, the compound may exhibit fluorescence turn-on under conditions of pH 6.5 to 8, and may detect homocysteine through fluorescence turn-on preferably under conditions of pH 7 to 8, more preferably under conditions of pH 7 to 7.8, and most preferably under conditions of pH 7.2 to 7.6.

In one embodiment, the compound may be a fluorescent probe compound which selectively emits fluorescence for cysteine or homocysteine, that is, may selectively detect cysteine or homocysteine in a sample containing at least one selected from the group consisting of biothiols, metal ions and biomolecules.

The sample may be water, a buffer solution, or a biological sample, and the biological sample may include cells, tissues, body fluids (saliva, etc.), blood, spinal fluid, cerebrospinal fluid, serum, plasma, urine, or feces isolated from humans or animals (rodents, mammals, etc.), and preferably, cells or tissues, but is not limited thereto, and may be any biological sample that may be used in the art.

In one embodiment, the compound may selectively emit fluorescence for cysteine or homocysteine by targeting tumors or cancers associated with cysteine or homocysteine.

In one embodiment, the tumors or cancers associated with cysteine or homocysteine may be tumors or cancers caused by the overexpression of cysteine or homocysteine, and may be, for example, at least one selected from the group consisting of brain tumor, colon cancer, stomach cancer, liver cancer, breast cancer, lung cancer, pancreatic cancer, gallbladder cancer, thyroid cancer, parathyroid cancer, kidney cancer, bladder cancer, prostate cancer, ovarian cancer, rectal cancer, central nervous system tumor, spinal cord tumor, cervical cancer, blood cancer, melanoma, colorectal cancer, bone cancer, skin cancer, head cancer, uterine cancer, vaginal cancer, vulvar carcinoma, esophageal cancer, small intestine cancer, and adrenal cancer. Preferably, the tumors or cancers may be at least one selected from the group consisting of brain tumor, liver cancer, cervical cancer, and skin cancer, more preferably, brain tumor, and most preferably, glioblastoma.

In an example of the present disclosure, as a novel fluorescent molecular probe compound capable of selectively detecting cysteine and homocysteine within a short period of time, a compound was designed and developed to configure 2-hydroxypyridine, an aromatic ring compound, as a functional group in 4-chloro-7-nitrobenzofurazan, thereby synthesizing and naming Compound 1 (NPO-o-Pyr) (see Example 1). In addition, it was confirmed that the compound of the present disclosure has stability in an aqueous solution (pH 7.4), which is a biological composition, and exhibits fluorescence turn-on near 550 nm when reacting with cysteine and homocysteine. In addition, since the compound contains 2-hydroxypyridine, a leaving group with low toxicity when treated to various cells, it was confirmed that the compound may overcome the toxicity problem of conventional fluorescent molecular probes and contrast agents (see Examples 2 to 7).

In addition, in the example of the present disclosure, it was confirmed that the compound of the present disclosure exhibited high targeting ability for tumors when applied to a glioblastoma xenograft mouse model, and showed a clear difference in fluorescence expression between glioblastoma tumor tissue and normal brain tissue (see Example 8). In addition, when treated in various forms (dipping form, spraying form, etc.) to glioblastoma clinical tumor tissue, the compound showed a fluorescence intensity capable of distinguishing a difference from normal brain tissue within 10 minutes (see Example 9). Through this, it was confirmed that the compound NPO-o-Pyr of the present disclosure may be applied for the use of imaging (contrast agent) for monitoring fluorescence-guided surgery to remove glioblastoma by confirming that the tumor site of glioblastoma may be distinguished using the compound, thereby completing the present disclosure.

In an aspect, the present disclosure relates to a fluorescent probe for detecting cysteine or homocysteine, including a compound having a leaving group in a structure represented by the following Chemical Formula 2, a hydrate thereof, a solvate thereof, or a pharmaceutically acceptable salt thereof.

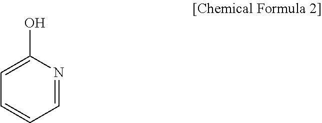

The leaving group in the structure represented by Chemical Formula 2 may be named 2-hydroxypyridine.

In one embodiment of the present disclosure, the compound having the leaving group in the structure represented by Chemical Formula 2 may be the compound represented by Chemical Formula 1.

In the fluorescent probe for detecting cysteine or homocysteine according to the present disclosure, the description of the compound represented by Chemical Formula 1 and the fluorescent probe is as described above.

In one embodiment of the present disclosure, the fluorescent probe may be able to target and image tumors or cancers associated with cysteine or homocysteine in vivo.

In one embodiment of the present disclosure, the fluorescent probe may be used in fluorescence-guided surgery for removing tumors or cancers.

In one embodiment of the present disclosure, the fluorescent probe of the present disclosure may selectively detect cysteine and/or homocysteine in a biological sample.

The biological sample may include cells, tissues, body fluids (saliva, etc.), blood, spinal fluid, cerebrospinal fluid, serum, plasma, urine, or feces isolated from humans or animals (rodents, mammals, etc.), and preferably, cells or tissues, but is not limited thereto, and may be any biological sample that may be used in the art.

In an aspect, the present disclosure relates to a composition for detecting cysteine or homocysteine, including the compound represented by Chemical Formula 1, a hydrate thereof, a solvate thereof, or a pharmaceutically acceptable salt thereof.

The composition for detecting cysteine or homocysteine of the present disclosure may selectively detect cysteine and/or homocysteine in a biological sample.

The composition for detecting cysteine or homocysteine of the present disclosure may further include at least one selected from a solvent, an acid, a base, and a buffer solution, and may be prepared by adding the above-described compound to a solvent, a buffer solution, or a mixture thereof, and adding an acid and/or a base thereto. In addition, the composition for detecting cysteine or homocysteine may additionally include other additives that may be used in the art. The contents of the solvent, the acid, the base, and the buffer solution included in the composition may be appropriately adjusted depending on the required performance. The solvent may include water, THE, methanol, ethanol, an aqueous HI solution, N,N-dimethylformamide, or a combination thereof. Alternatively, the composition may be mixed with a biological sample.

In the composition for detecting cysteine or homocysteine according to the present disclosure, the description of the compound represented by Chemical Formula 1 and the biological sample is as described above.

In an aspect, the present disclosure relates to a method for detecting cysteine or homocysteine, including treating a sample, a cell, a tissue or an organ with the compound represented by Chemical Formula 1 and irradiating an excitation light source; and measuring changes in emitted fluorescence.

In one embodiment of the present disclosure, the cell, tissue or organ may be cells, tissues or organs of tumors or cancers related to cysteine or homocysteine.

In the method for detecting cysteine or homocysteine according to the present disclosure, the description of the compound represented by Chemical Formula 1, the sample, and the tumors or cancers associated with cysteine or homocysteine is as described above.

In the method for detecting homocysteine according to the present disclosure, the excitation light source may be a light source having an excitation wavelength of 380 to 500 nm, preferably an excitation wavelength of 390 to 490 nm, and more preferably an excitation wavelength of 430 to 450 nm. Here, it is preferable to irradiate the light source using a fluorescence spectrophotometer or UV absorption spectroscopy.

In the method for detecting homocysteine according to the present disclosure, the measuring of changes in the emitted fluorescence may include confirming that changes in the absorption and fluorescence emission spectra occur by reacting the compound represented by Chemical Formula 1 according to the present disclosure selectively with cysteine and/or homocysteine. Preferably, the fluorescence emission and fluorescence intensity may be increased in the presence of cysteine and/or homocysteine.

In an aspect, the present disclosure relates to a composition for diagnosing diseases associated with cysteine or homocysteine, including the compound represented by Chemical Formula 1, a hydrate thereof, a solvate thereof, or a pharmaceutically acceptable salt thereof.

In the diagnostic composition according to the present disclosure, the description of the compound represented by Chemical Formula 1 and the tumors or cancers associated with cysteine or homocysteine is as described above.

In one aspect, the present disclosure relates to a method for providing information for diagnosing diseases associated with cysteine or homocysteine, including treating a tumor or cancer of a human or animal with the compound represented by Chemical Formula 1 (Step 1); irradiating an excitation light source to the tumor or cancer treated in Step 1 (Step 2); and measuring a change in emitted fluorescence (Step 3).

In the method for providing the information for diagnosis according to the present disclosure, the description of the compound represented by Chemical Formula 1, the excitation light source, and the tumors or cancers associated with cysteine or homocysteine is as described above.

In the method for providing the information for diagnosis according to the present disclosure, Step 3 includes a method for confirming that the fluorescence intensity of a tumor or cancer to be measured has increased compared to the fluorescence intensity before the compound is treated.

More specifically, Step 3 may include confirming that changes in absorption and fluorescence emission spectra occur by reacting the compound represented by Chemical Formula 1 according to the present disclosure selectively with cysteine and/or homocysteine. Preferably, the fluorescence turn-on and fluorescence intensity may increase in the presence of cysteine and/or homocysteine, and when the fluorescence emission and fluorescence intensity are increased compared to the tumor or cancer tissue before treatment with the compound according to the present disclosure, it may be determined that the tumor or cancer tissue and normal tissue may be distinguished.

In one aspect, the present disclosure provides a method for imaging a tumor or cancer tissue, including: adding the compound represented by Chemical Formula 1 to a tumor or cancer using cysteine and homocysteine as biomarkers (Step 1); irradiating an excitation light source to the tumor or cancer treated in Step 1 (Step 2); and measuring a change in emitted fluorescence (Step 3).

In the method for imaging the tumor or cancer tissue according to the present disclosure, the tumor or cancer using cysteine and homocysteine as the biomarkers may mean the same tumor or cancer as the above-mentioned tumors or cancers associated with cysteine or homocysteine.

In the role of the contrast agent for fluorescence-guided surgery capable of distinguishing and imaging tumors and cancers according to the present disclosure, the description of the compound represented by Chemical Formula 1, the excitation light source, and the tumors or cancers associated with cysteine or homocysteine is as described above.

The method for imaging the tumor and cancer tissue according to the present disclosure may target and image the tumor and cancer tissue by detecting cysteine and homocysteine present in the tumor and cancer.

In the method for imaging the tumors and cancer tissues according to the present disclosure, Step 3 may include confirming that changes in absorption and fluorescence spectra occur by reacting the compound represented by Chemical Formula 1 according to the present disclosure selectively with cysteine and homocysteine. Preferably, the fluorescence emission and fluorescence intensity may be increased by reacting with cysteine and homocysteine present in tumors and cancers.

In an aspect, the present disclosure relates to an imaging composition for monitoring fluorescence-guided surgery to remove tumors or cancers associated with cysteine or homocysteine, including the compound represented by Chemical Formula 1, a hydrate thereof, a solvate thereof or a pharmaceutically acceptable salt thereof.

In the imaging composition according to the present disclosure, the description of the compound represented by Chemical Formula 1 and the tumors or cancers associated with cysteine or homocysteine is as described above.

The imaging composition according to the present disclosure may be used in surgery to remove a glioblastoma tumor by detecting cysteine and homocysteine present in tumors or cancers associated with cysteine or homocysteine, preferably a glioblastoma tumor.

In an aspect, the present disclosure relates to a fluorescence-guided surgery method for removing tumors or cancers associated with cysteine or homocysteine including: administering to a patient the compound represented by Chemical Formula 1; illuminating a surgical site of the patient with an excitation light source to identify fluorescence emitted from the compound 1; and performing surgical resection of an area emitting the fluorescence.

In the imaging composition according to the present disclosure, the description of the compound represented by Chemical Formula 1, the excitation light source, and the tumors or cancers associated with cysteine or homocysteine is as described above.

Furthermore, the present disclosure provides a method for producing a compound represented by Chemical Formula 1, including reacting 4-(dimethylamino)pyridine, 2-hydroxypyridine, and 4-chloro-7-nitrobenzofurazan.

In the producing method according to the present disclosure, the reaction may be performed using N,N-dimethylformamide (DMF) deoxygenated with nitrogen as a reaction solvent.

Preferably, a first reaction may be performed by mixing 4-(dimethylamino)pyridine dissolved in DMF and 4-chloro-7-nitrobenzofuran dissolved in DMF and then a second reaction may be performed by adding 2-hydroxypyridine dissolved in DMF.

In the producing method according to the present disclosure, after the step, the method may further include a process of mixing ethyl acetate (EtOAc) and water in the reacted solution, and then separately recovering only an organic layer (EtOAc layer) and drying and concentrating the organic layer, and further include a process of purifying the organic layer using column chromatography or the like.

In the producing method according to the present disclosure, the producing process of the compound represented by Chemical Formula 1 may be represented as in Reaction Scheme 1 of Example 1 below.

### [Modes]

### Example 1. Synthesis and Structural Analysis of Fluorescent Molecular Probe Compound for Detecting Cysteine and Homocysteine for Tumor and Cancer Targeting

The present inventors designed a fluorescent molecular probe having high biocompatibility based on low toxicity capable of replacing contrast agents that may be used in fluorescence-guided surgery to remove tumors and cancers. More specifically, Compound 1 was designed to have 2-hydroxypyridine, an aromatic ring compound, as a functional group in 4-chloro-7-nitrobenzofurazan, and Compound 1 was synthesized according to the following Reaction Scheme.

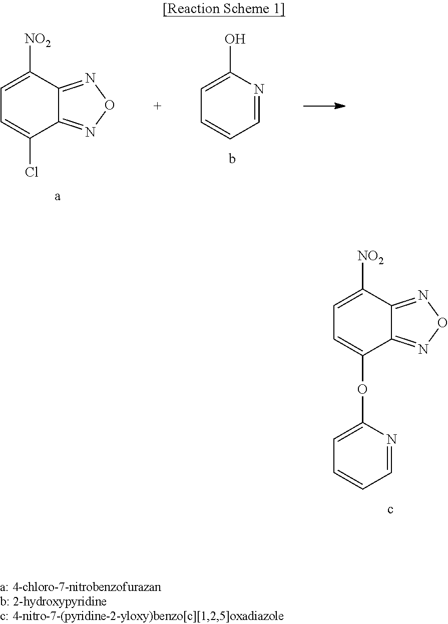 [SMILES: O=[N+]([O-])c1ccc(Cl)c2nonc12 ; Oc1ccccn1 ; O=[N+]([O-])c1ccc(Oc2ccccn2)c2nonc12]

Specifically, 4-(dimethylamino)pyridine (70.98 mg, 0.5810 mmol, SIGMA ALDRICH, 107700) was dissolved in 9 mL of N,N-dimethylformamide (DMF) deoxygenated with nitrogen, and the compound was well mixed and stirred at 25° C. for 10 minutes (revolutions per minute: 250 rpm) using a stirring bar. Thereafter, 4-chloro-7-nitrobenzofurazan (100 mg, 0.3873 mmol, SIGMA ALDRICH, 163260), a synthetic starting material, and 2 mL of N,N-dimethylformamide (DMF) deoxygenated with nitrogen were added to the solution, and the mixture was stirred at 60° C. for 10 minutes. Finally, the mixture was added with a solution of 2-hydroxypyridine (147.32 mg, 1.5492 mmol, SIGMA ALDRICH, 56800) dissolved in 3 mL of N,N-dimethylformamide (DMF), and then heated to 80° C. in a silicone oil container. When the temperature reached 80° C., the mixture was stirred for 60 minutes and then the reaction was stopped with water (10 mL). To extract the organic layer, ethyl acetate (EtOAc) and water were mixed in a 1:1 ratio using a separatory funnel, and through this process, only the organic layer could be obtained separately. Thereafter, the organic layer was dried with anhydrous sodium sulfate (Na₂SO₄, 5 g) and concentrated using a rotary evaporator under conditions of 37° C. and 20 to 500 mmHg. The dark brown solid compound thus obtained was separated (eluent: EA/n-Hexane=30/70) by using column chromatography (diameter 6 cm, height 15 cm) using silica gel (Merck-silica gel 60, 230 to 400 mesh) to obtain a yellow solid compound (Compound 1, 19.23 mg, 23.4%).

Nuclear magnetic resonance (NMR) analysis was performed to confirm whether the synthesis was successfully performed. Through the analysis, it was confirmed that Compound 1 was successfully synthesized. The NMR analysis results were as follows:

¹H-NMR (500 MHZ, DMSO-D6) δ 8.72, 8.70, 8.26, 8.26, 8.26, 8.25, 8.06, 8.06, 8.04, 8.03, 8.03, 7.45, 7.43, 7.42, 7.41, 7.38, 7.37, 7.36, 7.35, 3.30, 2.47, 2.47, 2.46, 2.46, 2.46, −0.03, −0.04, −0.05.

At this time, Compound 1 was identified as ‘4-nitro-7-(pyridine-4-yloxy)benzo[c][1,2,5]oxadiazole’, and named ‘NPO-o-Pyr’.

### Example 2. Confirmation of Fluorescence Emission Properties of Compound 1 (NPO-o-Pyr)

In order to confirm the fluorescence emission properties of Compound 1 (NPO-o-Pyr) prepared in Example 1, absorbance and fluorescence emission graphs were measured after reaction with cysteine and homocysteine in a neutral aqueous solution (pH 7.4, PBS buffer).

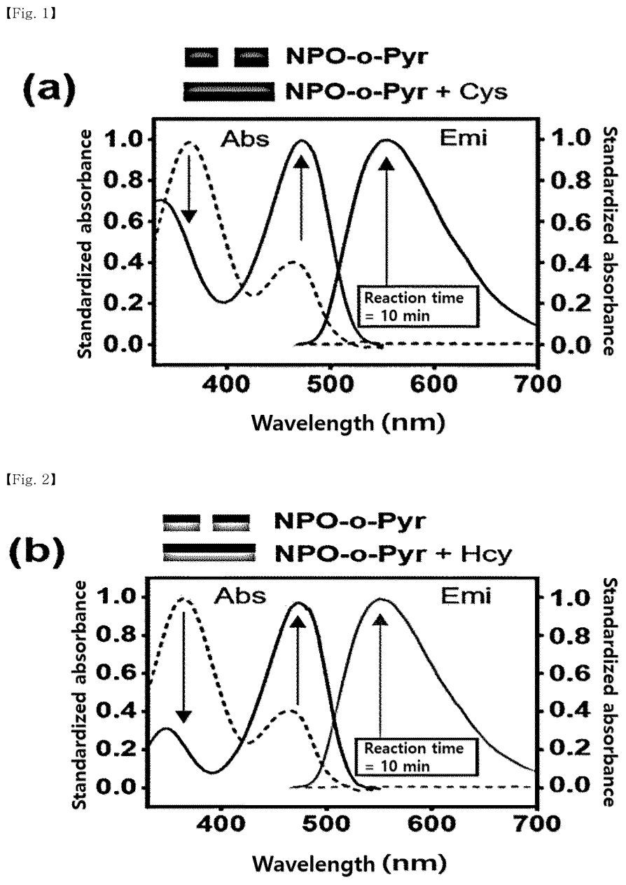

Specifically, Compound 1 (NPO0-o-Pyr, 10 μM), cysteine (L-cysteine, 10 μM), and homocysteine (DL-homocysteine, 10 M) were stirred in an aqueous solution (pH 7.4, PBS buffer) for 10 minutes, and then the fluorescence emission and absorbance values were measured (excitation wavelength: 430 nm). For analysis of absorption spectra (UV/Vis absorption spectra), a UV/Vis spectrophotometer (Agilent Technologies Cary 8454, USA) was used, and for analysis of the fluorescence spectra, a fluorescence spectra meter (SHIMADZU CORP. RF-6000, Japan) was used. At this time, a cell added with the compound in each device was used with a standard quartz cell (internal volume=0.1 cm) with a thickness of 1 cm. The measured results were shown in FIG. 1.

FIGS. 1 and 2 show results of changes in absorbance (Abs) and fluorescence emission (Emi) graphs according to a reaction of Compound 1 (NPO-o-Pyr) with cysteine and homocysteine under a neutral aqueous solution (pH 7.4, PBS buffer) condition. It was confirmed that the absorbance spectra increased at 488 nm and the fluorescence emission spectra increased at 550 nm. In addition, it was confirmed that Compound 1 (NPO-o-Pyr) exhibited fluorescence turn-on near 550 nm by reacting with cysteine and homocysteine.

### Example 3. Confirmation of Cysteine and Homocysteine Reaction Time and Selectivity of Compound 1 (NPO-o-Pyr)

To confirm whether Compound 1 (NPO-o-Pyr) exhibited a reaction with various types of aminothiols including cysteine and homocysteine under a neutral (pH 7.4) condition, Compound 1 (NPO-o-Pyr) and aminothiols were added to a neutral (pH 7.4) aqueous solution (pH 7.4, PBS buffer), and then the fluorescence emission graph was measured.

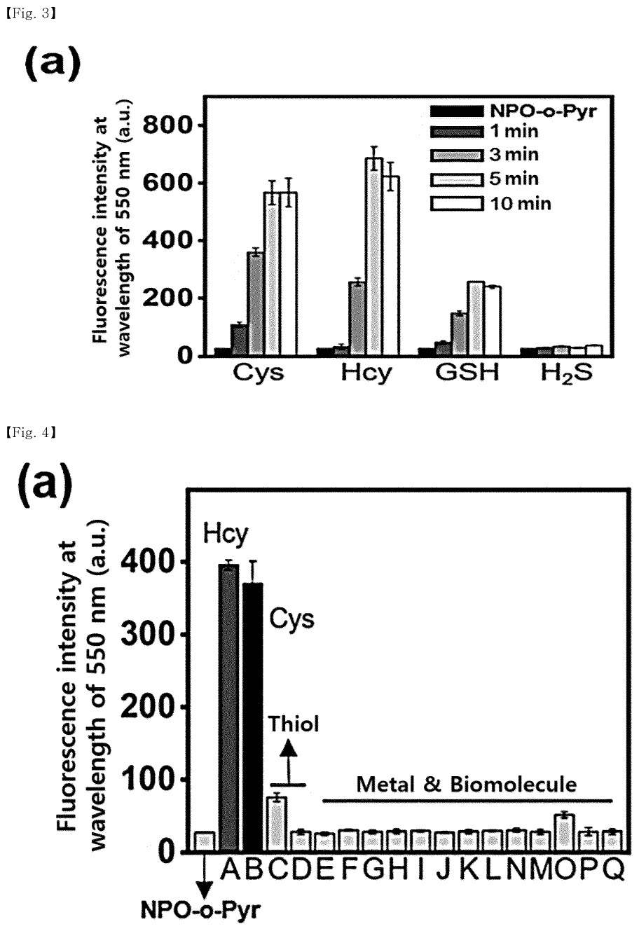

Specifically, Compound 1 (NPO0-o-Pyr, 10 μM) reacted with cysteine (L-cysteine, 10 μM), homocysteine (DL-homocysteine, 10 μM), glutathione (L-glutathione, 10 μM), and hydrogen sulfide (10 μM) in an aqueous solution (pH 7.4, PBS buffer), and the fluorescence values were measured (excitation wavelength: 430 nm). Aminothiols including homocysteine were used with products from Sigma and TCI, and a fluorescence photometer (SHIMADZU CORP, RF-6000, Japan) was used for analysis of fluorescence spectra. At this time, a cell added with the compound in each device was used with a standard quartz cell (internal volume=0.1 cm) with a thickness of 1 cm. The measured results were shown in FIG. 3.

FIG. 3 is a graph showing a reaction of Compound 1 (NPO-o-Pyr) and amino thiol over time (0 to 10 minutes) under an aqueous solution (pH 7.4, PBS buffer) condition. After adding cysteine and homocysteine, an increase in fluorescence of Compound 1 was observed within 3 minutes, and it was confirmed that the fluorescence intensity was maintained for up to 10 minutes, whereas no fluorescence emission was observed for other amino thiols except for cysteine and homocysteine to have low reactivity. Through this, it was confirmed that Compound 1 (NPO-o-Pyr) had the highest selectivity and a fast reaction time for cysteine and homocysteine among various amino thiols under an aqueous solution (pH 7.4, PBS buffer) condition.

### Example 4. Confirmation of Selectivity of Compound 1 (NPO-o-Pyr) for Biomolecules

Whether Compound 1 (NPO-o-Pyr) exhibited selectivity only for cysteine and homocysteine among various types of biomolecules was confirmed through fluorescence changes.

Specifically, the fluorescence turn-on was confirmed to confirm the selectivity of Compound 1 (NPO-o-Pyr) for cysteine and homocysteine. An aqueous solution (pH 7.4, PBS buffer) was used, and metal ions were used with products from Aldrich, Oriental Chemicals, Samchun chemicals, and Duksan chemicals. Compound 1 was dissolved in dimethyl sulfoxide (DMSO) at 10 mM and used, and the amount of dimethyl sulfoxide was controlled to be the same in each solvent (less than 1%) under a final used solvent condition. The concentration of Compound 1 was 10 μM, and the biomolecule was treated identically at 10 μM, an excitation wavelength of 430 nm was used, and the fluorescence values at a fluorescence wavelength of 550 nm were measured using a fluorescence spectrophotometer, and were shown in FIG. 4.

The graph in FIG. 4 is a graph showing the fluorescence intensity measured after treating various biological molecules and ions (anion) in an aqueous solution (pH 7.4, PBS buffer) containing Compound 1. In the graph of FIG. 4, a result of treating only Compound 1 was shown by a light gray bar graph, graphs of reacting cysteine (Cys) and homocysteine (Hcy) with Compound 1 were shown by dark gray bar graphs, and a result of reacting other biomolecules with Compound 1 was shown by a light gray bar graph. The types of ions indicated on the horizontal axis in the graph of FIG. 4 were as follows:
(A) Compound 1 and DL-Homocysteine (Probe 1 with DL-Homocysteine);
(B) Compound 1 and L-Cysteine (Probe 1 with L-Cysteine);
(C) Compound 1 and L-Glutathione (Probe 1 with L-Glutathion);
(D) Compound 1 and Hydrogen sulfide (Probe 1 with hydrogen sulfide);
(E) Compound 1 and Bovine serum albumin (Probe 1 with Bovine Serum Albumin);
(F) Compound 1 and Holmium (III) chloride (Probe 1 with Holmium (III) chloride);
(F) Compound 1 and Iron (II) chloride (Probe 1 with Iron (II) chloride);
(H) Compound 1 and Iron (III) chloride (Probe 1 with Iron (III) chloride);
(I) Compound 1 and Magnesium chloride (Probe 1 with Magnesium chloride);
(J) Compound 1 and Nikel (I) chloride (Probe 1 with Nikel (I) chloride);
(K) Compound 1 and Nikel (II) chloride (Probe 1 with Nikel (II) chloride);
(L) Compound 1 and Gold trichloride (Probe 1 with Gold trichloride);
(M) Compound 1 and Palladium (II) chloride (Probe 1 with Palladium (II) chloride);
(N) Compound 1 and Potassium chloride (Probe 1 with Potassium chloride);
(O) Compound 1 and Silver nitrate (Probe 1 with Silver nitrate);
(P) Compound 1 and Sodium chloride (Probe 1 with Sodium chloride); and
(Q) Compound 1 and Cobalt chloride (Probe 1 with Cobalt chloride).

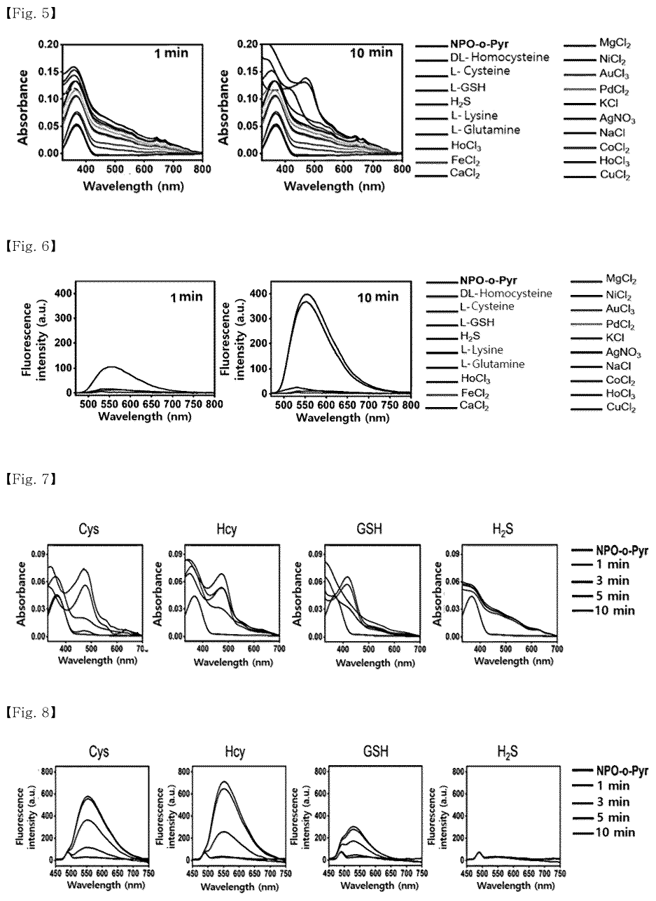

In addition, the absorbance and fluorescence spectra of the compounds were shown in FIGS. 5 and 6.

As a result, as shown in the graphs of FIGS. 4 to 6, Compound 1 itself (Only NPO-o-Pyr) showed almost no fluorescence intensity value, but A and B, where cysteine and homocysteine were present, respectively, showed strong fluorescence intensity values. In contrast, Compound 1 (NPO-o-Pyr) showed little fluorescence turn-on under conditions of other amino thiols except for cysteine and homocysteine, metal ions, and biomolecules.

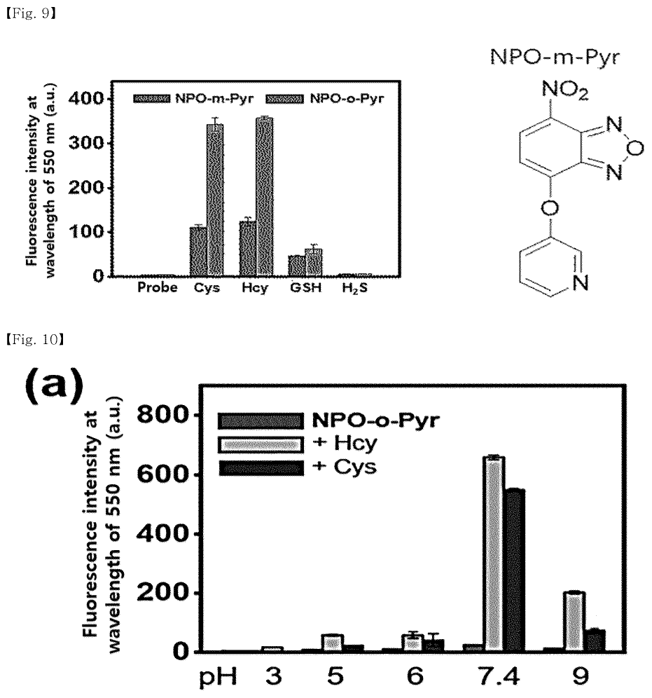

In addition, the selective efficacy over time for biothiol types such as cysteine, homocysteine, GSH, and H₂S was confirmed using the analysis method. The analysis results of the selectivity efficacy over time were shown in FIGS. 7 and 8.

As a result, as shown in FIGS. 7 and 8, in the case of GSH and H₂S, the fluorescence intensities were not significantly shown over time, but in the case of cysteine and homocysteine, the fluorescence intensities significantly increased over time.

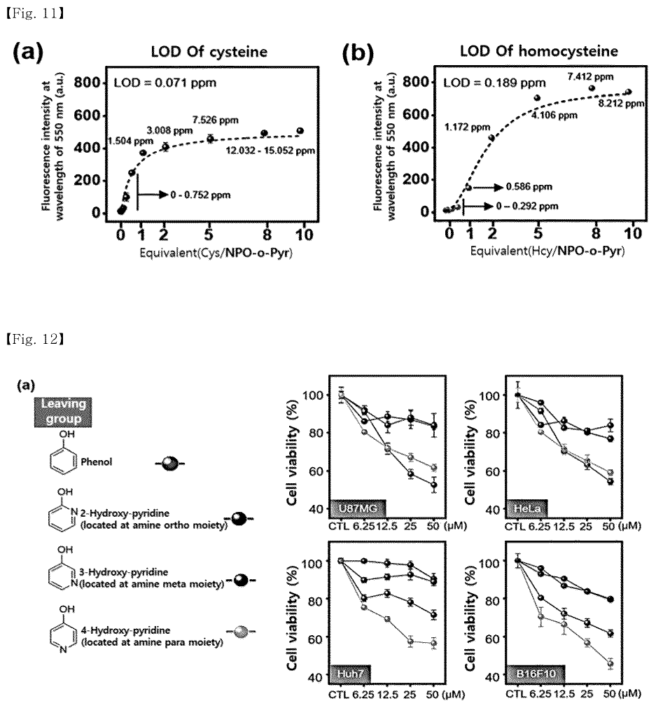

In addition, the selectivity of NPO-o-Pyr with NPO-m-Pyr (a substance with pyridine at a meta position rather than pyridine at an ortho position) synthesized as a control group was analyzed according to the analysis method. The analysis results were shown in FIG. 9.

As a result, as shown in FIG. 9, the control group, NPO-m-Pyr, showed a low fluorescence intensity, whereas Compound 1 showed a significantly high fluorescence intensity exceeding 300 in cysteine and homocysteine.

Through the results, it was confirmed that Compound 1 (NPO-o-Pyr) had high selectivity for cysteine and homocysteine.

### Example 5. Confirmation of Ability to Detect Cysteine and Homocysteine According to pH of Compound 1 (NPO-o-Pyr)

The present inventors confirmed the ability to detect cysteine and homocysteine of Compound 1 (NPO-o-Pyr) according to an acidity (pH).

Specifically, in order to observe the sensitivity of acidity (pH) for homocysteine based on the fluorescence turn-on of Compound 1, the sensitivity was confirmed under various acidity conditions (pH 3 to 9) while fixing the concentrations of Compound 1 (NPO-o-Pyr, 10 μM), cysteine (10 μM), and homocysteine (10 μM). That is, 10 μM of cysteine and homocysteine were treated under conditions of pH 3, 5, 7.4, and 9, respectively, and stirred for 10 minutes with Compound 1 (NPO-o-Pyr, 10 μM), and then the fluorescence intensity was measured. At this time, an excitation wavelength of 430 nm was used, and a fluorescence emission wavelength of 550 nm was measured, and the fluorescence intensity was shown in a bar graph in FIG. 10.

As a result, as shown in FIG. 10, it was confirmed that the most sensitive reactions of cysteine and homocysteine were observed at pH 7.4, and were relatively weak at other pHs, and thus it was confirmed that Compound 1 (NPO-o-Pyr) may be applied to the detection of cysteine and homocysteine in an aqueous solution state under conditions similar to a living organism.

### Example 6. Confirmation of Detection Limits to Cysteine and Homocysteine of Compound 1 (NPO-o-Pyr)

The detection limit values were analyzed to confirm the ability of Compound 1 (NPO-o-Pyr) to detect cysteine and homocysteine under conditions similar to an in vivo pH (pH 7.4).

The cysteine and homocysteine were treated at different concentrations (1 to 100 ng/μL) in the presence of Compound 1 (NPO-o-Pyr, 10 μM) using an aqueous solution (pH 7.4, PBS buffer), and then stirred at 37° C. for 10 minutes, and the fluorescence spectra were measured (excitation wavelength: 430 nm). Compound 1 was dissolved in a DMSO solution at 10 UM and used, and the amount of DMSO was prepared to be the same for each container (less than 1%) under the final solvent conditions used.

As a result, as shown in FIG. 11, it was confirmed that Compound 1 (NPO-o-Pyr) had a high ability to detect cysteine and homocysteine even at low concentrations. The LOD values were calculated as 0.071 ppm (=0.004 μM) for cysteine and 0.189 ppm (=0.011 μM) for homocysteine. Through these results, it was confirmed that the ability to detect cysteine and homocysteine, biomarkers of glioblastoma, was very excellent and there was a potential as a contrast agent for fluorescence-guided surgery.

### Example 7. Toxicity Analysis of Various Leaving Groups Including Leaving Group of Compound 1 (NPO-o-Pyr)

A leaving group generated after Compound 1 (NPO-o-Pyr) reacted with cysteine and homocysteine in vivo was 2-hydroxypyridine, and in order to use Compound 1 of the present disclosure as a contrast agent alternative in vivo, it was required to confirm biocompatibility. Therefore, the toxicity of the leaving group (2-hydroxypyridine) that may be generated after Compound 1 (NPO-o-Pyr) reacted with cysteine and homocysteine at various cells was evaluated.

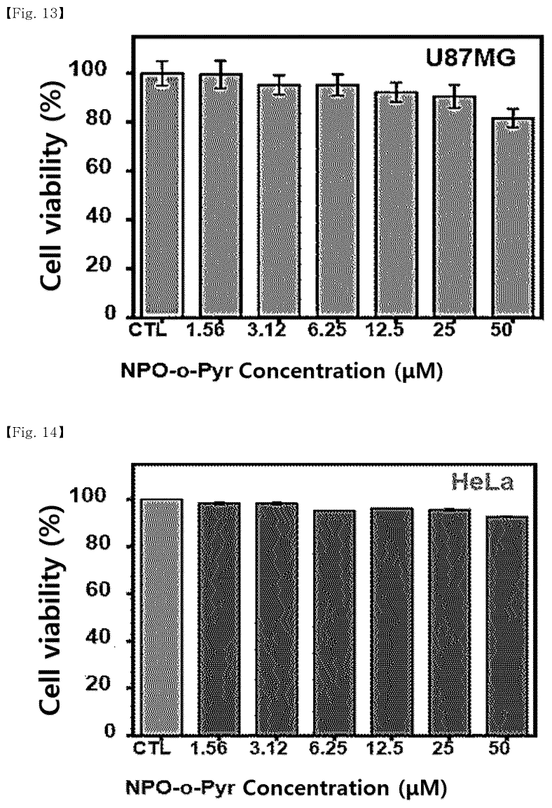

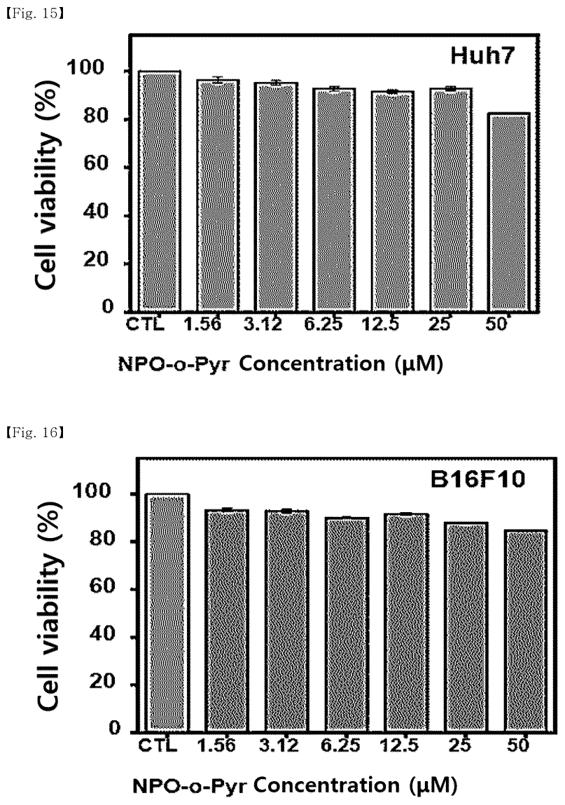

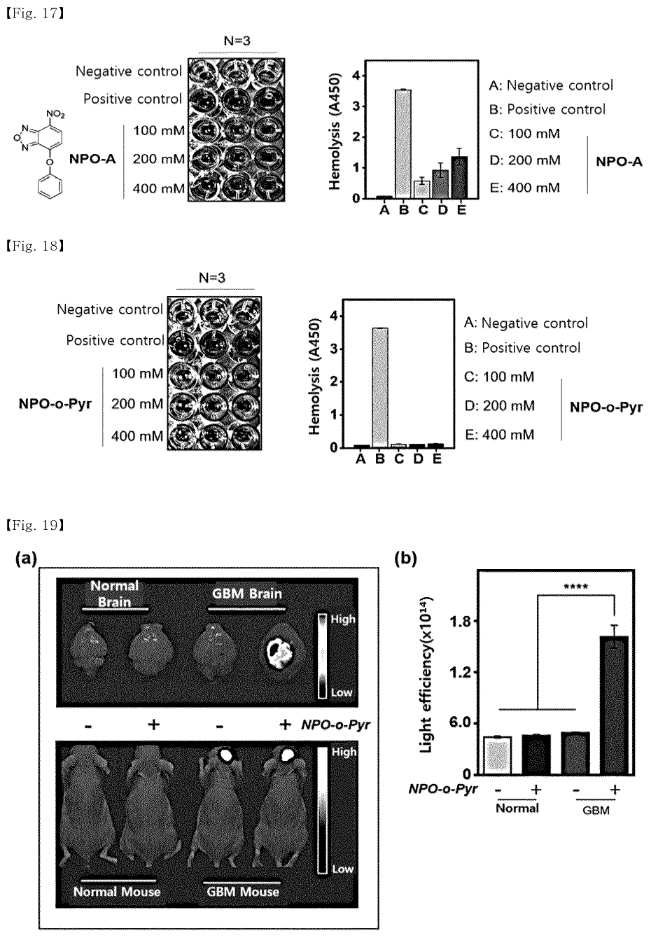

Specifically, for cell preparation, approximately 5×10³ cells each of glioblastoma cells (U87MG), hepatoma cells (Huh7), cervical cancer cells (HeLa), and skin cancer cells (B16F10) were treated in a 96-well clear plate (SPL Life Science, Ref. of Korea), cultured at 37° C. for 24 hours, and then treated with Compound 1 (NPO-o-Pyr, 10 μM) and analyzed using a CCK-8 assay kit (Dofindo, Japan) according to the manufacturer's instructions. Absorbance levels were analyzed at a wavelength of 490 nm by a microplate reader (SpctraMax Gemini EM, Molecular Devices, US). 2-Hydroxypyridine and various leaving groups with similar structures thereto were treated in U87MG, Huh7, HeLa, and B16F10 cells, and the cell viability was measured, which was shown in FIG. 12. The cell viability of U87MG, Huh7, HeLa, and B16F10 cells according to the concentration of Compound 1 was shown in FIGS. 13 to 16.

As a result, as shown in FIGS. 12 to 16, it was confirmed that the toxicity of the leaving group (2-hydroxypyridine) contained in Compound 1 (NPO-o-Pyr) was very low compared to the toxicity of leaving groups with similar structures, which indicated that the fluorescent probe of NPO-o-Pyr itself was biocompatible based on its low toxicity.

### Example 8. Hemolysis Analysis of Compound 1 (NPO-o-Pyr)

To evaluate an in vivo toxicity reaction of Compound 1, hemolysis of red blood cells was Confirmed when Compound 1 was Treated.

Blood collected from the heart of a mouse was treated with an anticoagulant and centrifuged at 2000 rpm for 5 minutes using a centrifuge to separate only blood cells. The separated blood cells were added with PBS and centrifuged again at 2000 rpm for 5 minutes to extract the blood cells. Only 8% of the total volume of the extracted blood cells was treated, treated with NPO-o-Pyr and NPO-A at 100, 200, and 400 mM, respectively, cultured at 37° C. for 1 hour, and then centrifuged again at 2000 rpm for 5 minutes to separate the supernatant. The absorbance of the separated supernatant was confirmed at 450 nm. A negative control group was set by treating red blood cells with only PBS, and a positive control group was set by treating red blood cells with 0.5% Triton X-100.

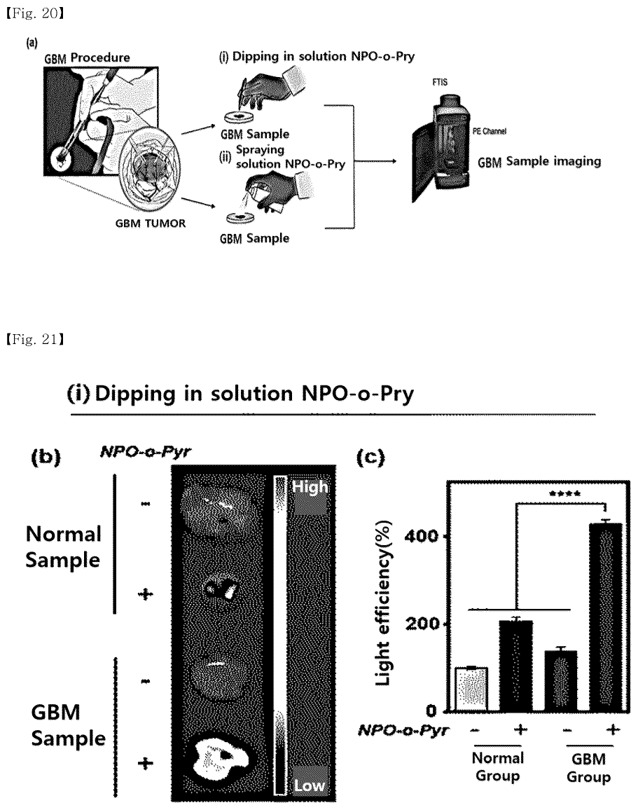

As a result, as shown in FIGS. 17 and 18, when red blood cells were treated with NPO-A, the degree of hemolysis increased as the concentration of NPO-A increased. However, in the case of Compound 1 (NPO-o-Pyr), hemolysis was found to be almost non-existent, similarly to the negative control group. Through the results, it was confirmed that Compound 1 (NPO-o-Pyr) was biocompatible and had lower cytotoxicity than conventional compounds.

### Example 9. Application of Compound 1 (NPO-o-Pyr) to Glioblastoma Xenograft Mouse Model

To determine whether mouse brain imaging was enabled when Compound 1 was applied to a glioblastoma xenograft mouse model, a normal mouse, a glioblastoma-induced control mouse (GBM mouse), and an experimental group administered with Compound 1 (NPO-o-Pyr) to each mouse were prepared, and brain imaging was confirmed.

First, in order to prepare the experimental group, LUC-U87MG cells (glioblastoma cells) were used. A solution containing 5.0×10⁵ cells in 5 μL of cell culture media from which fetal bovine serum (FBS) was removed was stereotaxically administered using a Hamilton microsyringe and then administered into the thalamus (−3.0 mm anteroposterior (AP), −1.8 mm medial-lateral (ML), and −3.0 mm dorsoventral (DV)) of the mouse brain anesthetized with isoflurane (1 μL/min, 5 min) to fabricate a glioblastoma (GBM) mouse model.

Thereafter, in order to determine the extent of glioblastoma progression through mouse brain imaging, luciferin (D-Luciferin, 150 mg/kg, n=10) was administered intraperitoneally to a normal mouse or a glioblastoma-induced mouse, and glioblastoma fluorescence emission signals (intensities) were confirmed using a fluorescence tissue imaging system (FTIS; VISQUE In Vivo Elite) (PE Channel: 390-490 nm excitation, 575-640 nm detection).

The results of mouse brain imaging for the normal group (Normal), the control group (GBM), and the experimental group administered with Compound 1 to each treatment group were shown in FIG. 19.

As a result, as shown in FIG. 19, when Compound 1 (NPO-o-Pyr) was administered via the tail vein to a glioblastoma xenograft mouse, it was confirmed that the fluorescence intensity increased in the tumor of the glioblastoma xenograft mouse model. That is, it was confirmed that Compound 1 could target and image the glioblastoma tumor when applied to the glioblastoma xenograft mouse model.

Through these results, it was confirmed that Compound 1 (NPO-o-Pyr) had a very excellent ability to target the glioblastoma tumor and had the potential to replace a contrast agent for fluorescence-guided surgery used to remove tumors and cancers, including glioblastoma, but not limited to glioblastoma.

### Example 10. Application of Compound 1 (NPO-o-Pyr) to Glioblastoma Clinical Tissue

By comparing cases where Compound 1 (NPO-o-Pyr) according to the present disclosure was applied to an actual glioblastoma clinical tissue sample and a normal brain tissue sample in various forms (dipping form, spraying form), it was confirmed whether glioblastoma tissue showed a difference in fluorescence intensity from normal brain tissue.

First, in order to remove impurities from the glioblastoma tumor (GBM tumor) tissue sample and the normal brain tissue sample obtained from actual clinical trials, each sample was dipped in 10 mL of an aqueous solution (phosphate-buffered saline; PBS, pH 7.4) and washed three times by stirring for 10 minutes in a shaker at 37° C. Thereafter, the concentration of Compound 1 (NPO-o-Pyr) was fixed at 100 UM and treated on each sample in two forms of dipping and spraying. In the case of the dipping form, each sample was dipped in a fixed concentration of Compound 1 (NPO-o-Pyr) and stirred at room temperature for 1 hour. In the case of the spraying form, the sample was sprayed with an appropriate amount of a fixed concentration of Compound 1 (NPO-o-Pyr) and left at 25° C.

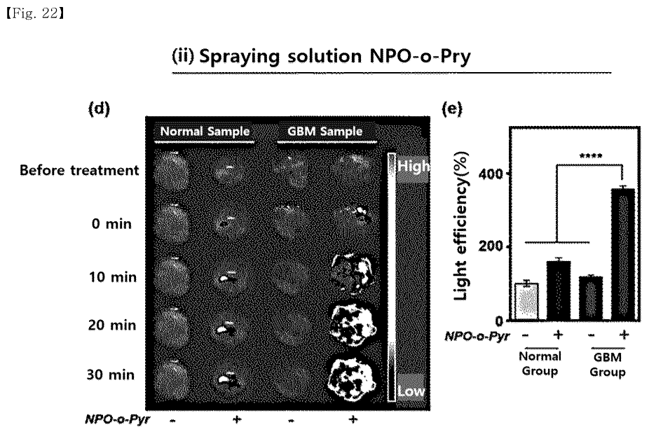

Subsequently, in order to determine a difference in fluorescence intensity between the glioblastoma tumor tissue sample and the normal brain tissue sample, the fluorescence emission signal (intensity) of each sample was confirmed using a fluorescence tissue imaging system (FTIS; VISQUE In Vivo Elite. Vieworks) (PE Channel: 390-490 nm excitation, 575-640 nm detection). A schematic diagram showing the analysis process was shown in FIG. 20.

As a result, as shown in FIG. 21, the dipping form showed a clear difference in fluorescence intensity between the glioblastoma tumor tissue sample and the normal brain tissue sample. In addition, as shown in FIG. 22, the difference in fluorescence intensity over time was observed even in the spraying form. As these results, it was shown that Compound 1 (NPO-o-Pyr) had a very excellent ability to target the glioblastoma tumor, and had a high targeting ability for tumors and cancers including glioblastoma, but not limited to the glioblastoma tumor.

The aforementioned description of the present disclosure is used for exemplification, and it may be understood by those skilled in the art that the present disclosure may be easily modified in other detailed forms without changing the technical spirit or requisite features of the present disclosure. Therefore, it should be appreciated that the aforementioned examples are illustrative in all aspects and are not restricted.

1. A compound represented by the following Chemical Formula 1:

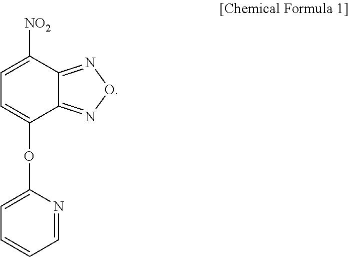 [SMILES: O=[N+]([O-])c1ccc(Oc2ccccn2)c2nonc12]

2. The compound of claim 1, wherein the compound reacts with cysteine or homocysteine to exhibit fluorescence turn-on.

3. The compound of claim 2, wherein the compound exhibits fluorescence turn-on under conditions of pH 6.5 to 8.

4. The compound of claim 1, wherein the compound selectively emits fluorescence for cysteine or homocysteine in a sample containing at least one selected from the group consisting of biothiols, metal ions and biomolecules.

5. The compound of claim 1, wherein the compound selectively emits fluorescence for cysteine or homocysteine by targeting tumors or cancers associated with cysteine or homocysteine.

6. The compound of claim 1, wherein the tumors or cancers are one or more selected from the group consisting of brain tumor, colon cancer, stomach cancer, liver cancer, breast cancer, lung cancer, pancreatic cancer, gallbladder cancer, thyroid cancer, parathyroid cancer, kidney cancer, bladder cancer, prostate cancer, ovarian cancer, rectal cancer, central nervous system tumor, spinal cord tumor, cervical cancer, blood cancer, melanoma, colorectal cancer, bone cancer, skin cancer, head cancer, uterine cancer, vaginal cancer, vulvar carcinoma, esophageal cancer, small intestine cancer, and adrenal cancer.

7. The compound of claim 1, wherein the compound having a leaving group in a structure represented by the following Chemical Formula 2:

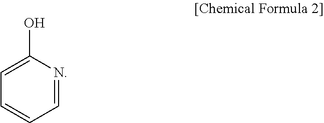

8. A method for detecting cysteine or homocysteine comprising:
treating a sample, a cell, a tissue or an organ with the compound of claim 1 and irradiating an excitation light source; and
measuring changes in emitted fluorescence.

9. The method for detecting cysteine or homocysteine of claim 8, wherein the cell, tissue or organ is cells, tissues or organs of tumors or cancers associated with cysteine or homocysteine.

10. A method for producing a compound represented by the following Chemical Formula 1, comprising reacting 4-(dimethylamino)pyridine, 2-hydroxypyridine, and 4-chloro-7-nitrobenzofurazan: 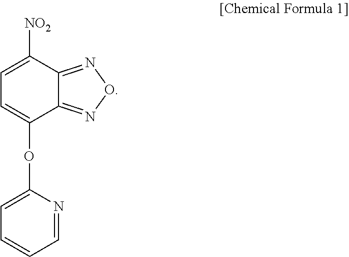 [SMILES: O=[N+]([O-])c1ccc(Oc2ccccn2)c2nonc12]
